(19) United States
(12) Patent Application Publication
(10) Pub. No.: US 2003/0168358 A1
(43) Pub. Date: Sep. 11, 2003

(54) Single-dose bend package

(75) Inventors: Christian Bressler, Frankfurt (DE); Markus Balkenhol, Neuberg (DE)

Correspondence Address: WILLIAM GERSTENZANG, NORRIS, MCLAUGHLIN & MARCUS, P.A., 220 EAST 42ND STREET, 30TH FLOOR, NEW YORK, NY, 10017, US

(73) Assignee: Heraeus Kulzer GmbH & Co., KG, Hanau (DE)

(21) Appl. No.: 10/369,064

(22) Filed: Feb. 18, 2003

(30) Foreign Application Priority Data
Feb. 18, 2002 (DE) 102 06 874.4

(51) Int. Cl. A61B 19/02

(52) U.S. Cl. 206/63.5; 206/764; 206/775

(57) ABSTRACT

A package for individual doses of a liquid, semi-solid, or pasty material, in the form of a card, which can be folded and converted to a container that can stand on its own.

The invention relates to a package for individual doses of a liquid, semi-solid, or pasty material in the form of a card, which can be folded and converted to a container that can stand on its own.

### BACKGROUND OF THE INVENTION

Liquid dental materials are frequently applied to teeth being treated using dispensing or application instruments, such as paintbrushes or bristle brushes (e.g. a so-called “Microbrush®”). In this, it is important that the application be simple and that the liquid can be removed from the container and applied using only one hand. This is less of a problem if the material is to be removed from a flask. Frequently, however, dental materials are supplied in so-called “single dose” packages, in which each time a single portion is freshly opened, protecting the remaining portions against spoilage or contamination. Small packages of this kind must usually be either placed in suitable supportive stands, or manipulated using two hands.

### SUMMARY OF THE INVENTION

In view of this problem, a new approach has been chosen, and another type of single-dose package has been found, which has the following characterizing features:

The form of a card having at least one straight side,

a straight fold line, parallel to one side of the card,

a window in the area of the fold line that holds a material container,

an apparatus for locking the card in its folded state,

an apparatus for opening the material container,

and as a special feature, the fact that folding the card creates an open standing container, allowing the removal of the material using a dispensing instrument, wherein, when the card is folded, the material container is oriented horizontally, with free access at the top.

This type of package is of course suitable for use with any type of liquids, pastes or semi-solid materials, for which single-dose packaging makes sense. These are especially liquids that become easily dried out or become encrusted around the container opening when held in resealable containers. This is problematic, for example, in the case of nail polish, correction fluid, mascara, and artists' paints. It is also possible to fill cyanoacrylate adhesives or 2-component adhesives into the package specified in the invention. The package is also well suited for paste material that is applied in small doses, which would be removed from the package using a spatula, for example.

### DETAILED DESCRIPTION

The card is preferably rectangular or square in shape, but may also take on other shapes that have at least one straight side, e.g. a trapezoid or a triangle, as long as the symmetry will allow the package to stand in a stable fashion once it has been folded.

The card is preferably made of a plastic. The card can be folded parallel to its straight side, along a straight fold line. This can be effected, for example, through a notch that is included on one side of the card. It is further provided that the card can be locked in a folded position and—in the case of a rectangular or square card—can be stood up in the manner of a roof. A possible locking mechanism can be provided by a click-stop or snap device, such as a plastic tongue, which would become latched in an opening in the card. In the area of the fold line, the card is equipped with a window, in which a material container is positioned. After the card has been folded and stood up, this material container can be opened in some appropriate fashion—for example by removing a covering foil. The user can then remove the material or the liquid using a suitable dispensing instrument, such as a brush, Microbrush, spoon, or spatula.

It can be provided that the corresponding instrument is attached to the package such that it can be easily removed.

It is not necessary for the fold line to lie precisely at the center of the card. Even if it is shifted somewhat toward the side, the card will be able to stand up.

The material container (tank) can be designed as follows, for example:

(a) The tank is formed by the plastic component (card), wherein a recessed opening in the card is designed to hold a liquid. This opening is sealed by a foil, or

(b) by another plastic component in the form of a cover.

Advantageously, in a) and b) the foil or the cover is connected to the card such that when the card is folded it is opened. In the case of the foil, this can be affected by providing a tongue (_\hat{ }_)or a tab (_Ω_) along the fold line, which lifts or pushes up the foil on the card when the card is folded.

Of course, the foil may also be provided with an overhang of sufficient length, as is shown in FIG. 5, which can be torn off by hand.

c) The tank is formed by a recessed area in the card, designed to hold the material, and sealed on both sides with foil.

In the case of c), it is again useful for one of the two sealing foils to be provided with a means for opening the tank when the card is folded (as described in reference to b)).

d) The tank is formed by two foils, which are sealed along their sides (known to experts in the field as a 4-side-seal pouch) or by

e) a foil that is sealed along 3 sides (3-side-seal pouch).

In the case of the side-sealed pouches d) and e), the method of opening is usefully such that when the card is folded, a section of the pouch along the seal is pulled off like a cover.

Advantages of the package specified in the invention are as follows, for example: its ability to stand up independently once it is folded; it is equally well-suited for use by right-handed and left-handed users; it can be manipulated with one hand in the removal of liquid materials; and no separate stand is necessary.

Below, the invention will be described in greater detail using the diagrams of a single-dose package for dental materials, without serving to limit the scope of the invention to this embodiment:

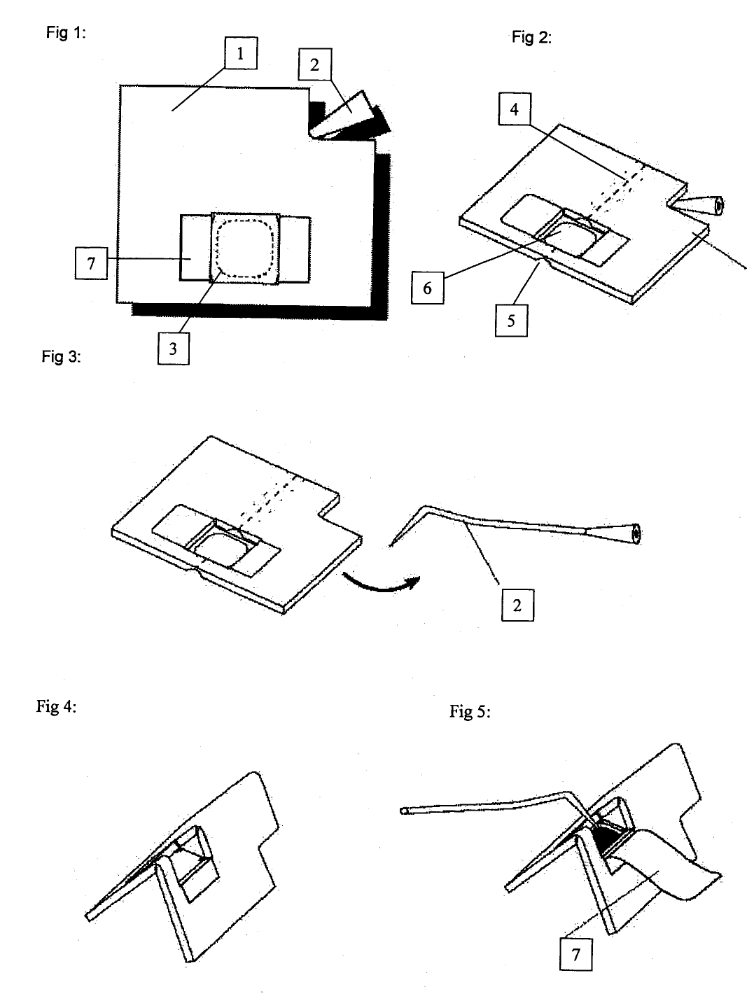

FIG. 1 shows an overhead view of the rectangular card in an unfolded position (as-delivered condition),

FIG. 2 shows an oblique view of the card,

FIG. 3 illustrates the removal of the Microbrush,

FIG. 4 shows the card in a folded, standing position, prior to the opening of the material container, and

FIG. 5 shows the card after the foil covering on the material container has been turned upward.

FIG. 1 shows an overhead view of the card 1 before it has been folded (as-delivered condition). At the upper right corner is a recess, to which a Microbrush 2 is attached such that it can be easily removed. In this diagram, the window with the material container 3 is sealed by a foil 7. The shape of the material container 3 is indicated by a dashed line.

FIG. 2 shows an oblique view of the card. The notch 5 is visible along the dashed fold line 4. In this view, the window 6 for the material container 3 is shown open.

FIG. 3 shows the removal of the Microbrush 2, which was covered in FIGS. 1 and 2.

FIG. 4 shows the card once it has been folded, prior to the opening of the material container 3. The locking, for example via a stop-catch device, is not shown in this diagram. The horizontal orientation of the material container and its free accessibility is shown in the upper section.

FIG. 5 shows the card after the covering foil 7 for the material container 3 has been turned upward. Further, the removal of the material (black surface) with the Microbrush 2 is shown.

The above-described package is preferably used for dental materials.

We claim:

1. Single-dose package for liquid, pasty, or semi-solid materials, in the form of a card (1) having
at least one straight side,
a straight fold line (4) aligned parallel to one side of the card,
a window (6) in the area of the fold line, containing a material container (3),
a lock for securing the card in a folded state,
wherein, when the card is folded, a free-standing container for removal of the liquid is formed, and
wherein once the card has been folded and stood up, and the material container opened, the material container (3) is oriented horizontally and is freely accessible from the above.

2. Package according to claim 1, wherein a sealing foil (7) that can be pulled off is provided on the material container (3) as the means of opening said container.

3. Package according to claim 1, wherein a notch (5) is included on one side of the card along the straight fold line (4).

4. Package according to claim 1, wherein said lick is a stop-catch or snap device.

5. Package according to claim 1, wherein a recess is provided at one corner, to which a dispensing instrument is removably attached.

6. Package pursuant to claim 5, wherein said dispensing instrument is selected from the group consisting of brush, spatula, spoon, and microbrush.
